(19) United States
(12) Patent Application Publication
(10) Pub. No.: US 2008/0114435 A1
(43) Pub. Date: May 15, 2008

(54) Flexible delivery system

(75) Inventor: Jason Samuel-Horner Bowe, West Lafayette, IN (US)

Correspondence Address: BRINKS HOFER GILSON & LIONE/CHICAGO/COOK, PO BOX 10395, CHICAGO, IL, 60610, US

(73) Assignee: MED Institute, Inc., West Lafayette, IN (US)

(21) Appl. No.: 11/715,078

(22) Filed: Mar. 7, 2007

Related U.S. Application Data
(60) Provisional application No. 60/779,815, filed on Mar. 7, 2006.

(51) Int. Cl. A61F 2/84

(52) U.S. Cl. 623/1.11

(57) ABSTRACT

A delivery system for an intraluminal medical device is disclosed. The delivery system comprises an elongate tubular sheath and an elongate tubular pusher. The pusher is slidably disposed within a lumen of the sheath and comprises at least one generally helical score in an exterior surface thereof for providing enhanced flexibility to the pusher.

### RELATED APPLICATIONS

This application claims the benefit of the filing date under 35 U.S.C. §119(e) of U.S. Provisional Patent Application Ser. No. 60/779,815, filed Mar. 7, 2006, which is incorporated herein by reference.

### BACKGROUND OF THE INVENTION

1. Field of the Invention

This invention relates to a medical device and, in particular, to a system for delivering a medical device.

2. Description of Related Art

The deployment of a medical device, such as an intraluminal prosthesis, into the lumen of a patient from a remote location by the use of a delivery device is generally known. Radially-expandable prostheses can be used, for example, to repair diseased and damaged aorta such as abdominal aortic aneurysms and thoracic aortic aneurysms. For example, a stent-graft may be loaded onto a delivery and deployment device and percutaneously inserted into the body lumen of a patient in a radially-compressed configuration. Once the prosthesis is in a proper position, it may be released so that it can radially expand to engage the walls of the body lumen. Exemplary expandable prostheses may be balloon-expandable, self-expanding, or both.

In general, delivery and deployment devices for intraluminal prostheses may include means for retaining and releasing the prosthesis into the body lumen. For example, such a device may include a cover or sheath for radially retaining the prosthesis in a compressed configuration. A pusher may be provided for pushing the sheath and the prosthesis into the body lumen and for delivering the device into a desired position. To deploy the prosthesis, the sheath may be withdrawn over the pusher and the prosthesis, thereby causing the prosthesis to become exposed and to expand into the body lumen.

There is currently a demand for delivery devices that are flexible and that are capable of negotiating or tracking complex and tortuous body lumina, for example the aortic arch. Such devices should exhibit high axial flexibility or trackability. One solution to improve the trackability of such delivery systems includes designing delivery system components out of generally flexible materials. For example, the sheath may comprise a generally flexible material, such as a low-durometer polyethylene or polytetrafluoroethylene (PTFE).

While it may be practical to use highly flexible materials for some applications, such as the introducer sheath, their use may be impractical for other applications, such as the pusher. The pusher must possess a high degree of pushability and therefore must, in general, possess high column strength, particularly when it is used to push a prosthesis within the introducer sheath. In general, soft and flexible materials may not possess sufficient structural integrity or strength for pusher applications. To this end, prior art pushers have been provided that are generally rigid and consequently have poor trackability.

There are many disadvantages of using a rigid pusher. For example, rigid pushers may preclude intraluminal intervention for patients with highly complex and tortuous body lumina. Also, rigid materials may possess poor kink-resistance and therefore may be susceptible to damage. The use of rigid pushers may also adversely affect the integrity of other system components, for example the sheath. For example, a relatively flexible sheath may bend or kink in the transition region between the prosthesis and a relatively rigid pusher. Accordingly, there is a present need in the art for an intraluminal prosthesis delivery and deployment system that addresses these and other problems.

### SUMMARY

According to an aspect of the present invention, a delivery system for an intraluminal medical device is provided and comprises an elongate tubular sheath and an elongate tubular pusher. The sheath has a proximal end, a distal end, and a sheath lumen. The pusher has a proximal end, a distal end, an exterior surface, and a lumen defining an interior surface and is slidably disposed within the sheath lumen.

The pusher may comprise at least one generally helical score in the exterior surface that provides enhanced flexibility to the pusher. The at least one helical score has a pitch that may be longitudinally uniform or that may be longitudinally variable. For example, the pitch may decrease proximally with the pusher, thereby providing the pusher with proximally-decreasing flexibility. The at least one score may have a depth that is less than or equal to the thickness of the pusher wall, where the wall thickness is defined by the interior and exterior surfaces of the pusher. The at least one score may extend proximally from the distal end of the pusher, or it may extend proximally from a position intermediate the proximal and distal pusher ends.

According to another aspect of the invention, a pusher may be provided that comprises a plurality of helical scores, where the scores are positioned symmetrically about the pusher.

According to another aspect of the invention, a pusher may be provided that comprises a first helical score having a proximal end and a distal end and a second helical score having a proximal end and a distal end. The first score may have a length that is equal to or greater than a length of the second score. At least one of the distal end and the proximal end of the first score may be coterminous with at least one of the distal end and the proximal end of the second score. For example, the distal ends of the scores may be longitudinally coterminous and/or the proximal ends of the scores may be longitudinally coterminous. Alternatively, the distal end of the first score may be longitudinally coterminous with the proximal end of the second score or vice versa.

According to another aspect of the invention, the system may comprise a radially-expandable intraluminal prosthesis disposed in a compressed configuration within a distal portion of the sheath lumen. In such embodiments, the pusher may be configured to push the prosthesis distally within the sheath lumen when the sheath is slid proximally in relation to the pusher.

According to another aspect of the invention, the distal end of the pusher may comprise a dilator having a tapered distal end. in such embodiments, the dilator extends distally from the sheath lumen and facilitates dilation of constricted vessels as the delivery system travels within a body lumen.

According to another aspect of the invention, a system may be provided and comprise an elongate inner sheath fixedly disposed within the pusher lumen between the proximal and distal pusher ends. The inner sheath preferably comprises a relatively soft and flexible plastic material, for example a low-durometer PTFE or nylon, whereas the pusher may comprise a relatively rigid material, for example a high-durometer nylon. In a preferred embodiment, the pusher comprises a material that is more rigid than the inner sheath material.

According to another aspect of the invention, a pusher may be provided that comprises a first material along a first portion thereof, and a second material along a second portion thereof where the first material is more flexible than the second material. The first portion and/or the second portion of the pusher may comprise at least one generally helical score in the exterior surface for providing enhanced flexibility to the second portion of the pusher.

According to another aspect of the invention, the system may comprise a limiting member for limiting the proximal displacement of the sheath relative to the pusher, thereby limiting distal retraction of the pusher from the sheath lumen.

According to another aspect of the invention, a system may be provided and comprise a guide cannula having a proximal end and a distal end disposed within the lumen of the pusher. The guide cannula may be configured to receive the expandable prosthesis over a distal portion thereof. Additional features include a haemostatic seal for controlling blood loss through the pusher and a guide cannula.

### BRIEF DESCRIPTION OF THE DRAWINGS

FIG. 1 is a perspective view of a delivery and deployment device according to an aspect of the present invention;

FIG. 2 is a sectional detail view of a portion of the delivery and deployment device of FIG. 1 around the proximal end of the prosthesis;

FIG. 3 is a sectional detail view of a portion of the delivery and deployment device of FIG. 1 around the distal end of the prosthesis;

FIG. 4 is a plan view of a distal retention device of the delivery and deployment device of FIG. 1;

FIG. 5A is a side perspective view of a pusher according to an aspect of the invention;

FIG. 5B is a side perspective view of a pusher according to an aspect of the invention;

FIG. 6 is a side perspective view of a pusher according to an aspect of the invention;

FIG. 7A is an end cross-sectional view of a pusher according to an aspect of the invention;

FIG. 7B is a sectional view of a pusher according to an aspect of the invention;

FIG. 7C is a sectional view of a pusher according to an aspect of the invention;

FIG. 7D is a cross-sectional view of a score according to an aspect of the invention;

FIG. 8 is a sectional view of a portion of the delivery and deployment device of FIG. 1 around a haemostatic seal;

FIG. 9 is a perspective view of a medial portion of the delivery and deployment device of FIG. 1;

FIG. 10 is a sectional view of a portion of the delivery and deployment device of FIG. 1 around the trigger wire release mechanism;

FIG. 11 is a sectional view of a portion of the delivery and deployment device of FIG. 1 around the pin vise clamp and the medical reagent introduction tube;

FIG. 12A is a perspective view of another delivery device according to an aspect of the invention;

FIG. 12B is a cross-sectional view of a distal portion of the delivery device of FIG. 12A;

FIG. 13 is a segmented sectional view of a delivery and deployment device that is fully loaded and ready for introduction into a patient;

FIG. 14 is a segmented sectional view of a delivery and deployment device demonstrating the prosthesis in an initial stage of deployment;

FIG. 15 is a segmented sectional view of a delivery and deployment device demonstrating the release of the prosthesis distal end during deployment;

FIG. 16 is a segmented sectional view of a delivery and deployment device demonstrating the release of the prosthesis proximal end during deployment; and

FIG. 17 is a segmented sectional view of a delivery and deployment device demonstrating the device in a configuration for withdrawal from the body lumen.

### DETAILED DESCRIPTION OF THE PREFERRED EMBODIMENTS

Throughout the specification, the terms “distal” and “distally” shall denote a position, direction, or orientation that is generally toward the patient. Accordingly, the terms “proximal” and “proximally” shall denote a position, direction, or orientation that is generally away from the patient.

Throughout the specification, unless the context requires otherwise, the words “comprise” and “include” and variations such as “comprising” and “including” will be understood to imply the inclusion of an item or group of items, but not the exclusion of any other item or group of items.

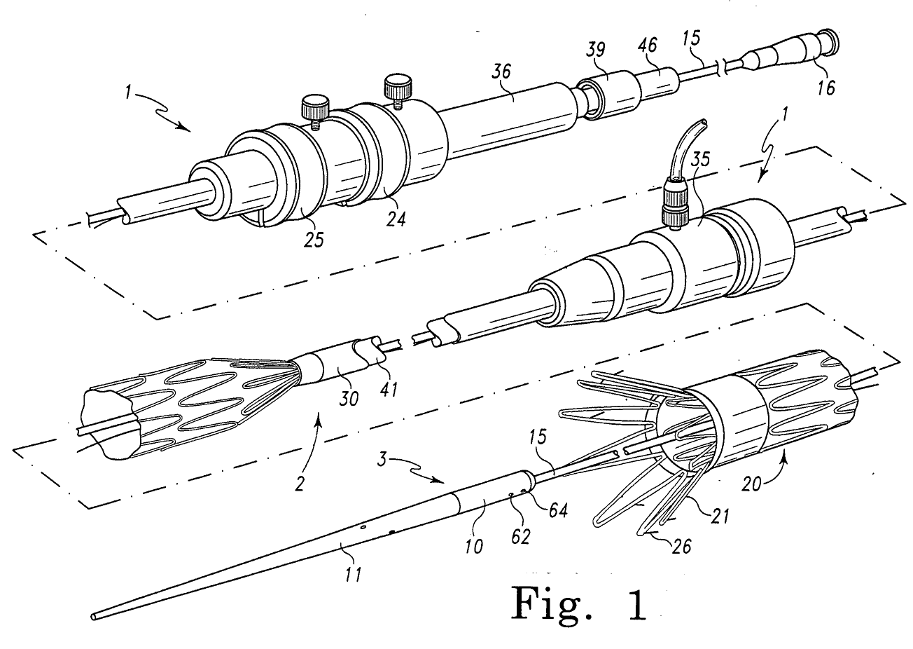

FIG. 1 shows a delivery system for an intraluminal medical device; in particular, a system for delivering and deploying an intraluminal prosthesis 20 in a lumen of a patient during a medical procedure. The system includes an external manipulation section 1, a proximal positioning mechanism or attachment region 2, and a distal positioning mechanism or attachment region 3. During a medical procedure to deploy the prosthesis 20, the proximal and distal attachment regions 2 and 3 will travel through the lumen to a desired deployment site. The external manipulation section 1, which is acted upon by a user to manipulate the delivery and deployment device, remains outside of the patient throughout the procedure.

The prosthesis 20 may comprise a tubular graft material, such as Dacron. The prosthesis 20 may additionally or alternatively comprise a stent 19. The stent 19 may be self-expanding and cause the prosthesis 20 to expand when released from the delivery and deployment device. The stent 19 may be coupled to an interior or an exterior surface of the graft material. The prosthesis 20, as shown in FIG. 1, comprises a graft material and a plurality of self-expanding stents 19. The prosthesis comprises at least one self-expanding stent 19 disposed on an interior surface of the graft material, and at least one self-expanding stent 19 disposed on an exterior surface of the graft material.

The prosthesis 20 may optionally include a bare wire stent 21 disposed on an end of the prosthesis. The bare wire stent 21 expands and engages the body lumen, thereby anchoring the prosthesis 20 and preventing the prosthesis from moving after implantation. As shown in FIG. 1, the stent 21 includes a self-expanding zigzag stent. The stent 21 may comprise anchoring means, for example barbs 26, that are configured to grasp the walls of the body lumen. Stents 19, 21 may comprise any suitable biocompatible material, including stainless steel and nitinol.

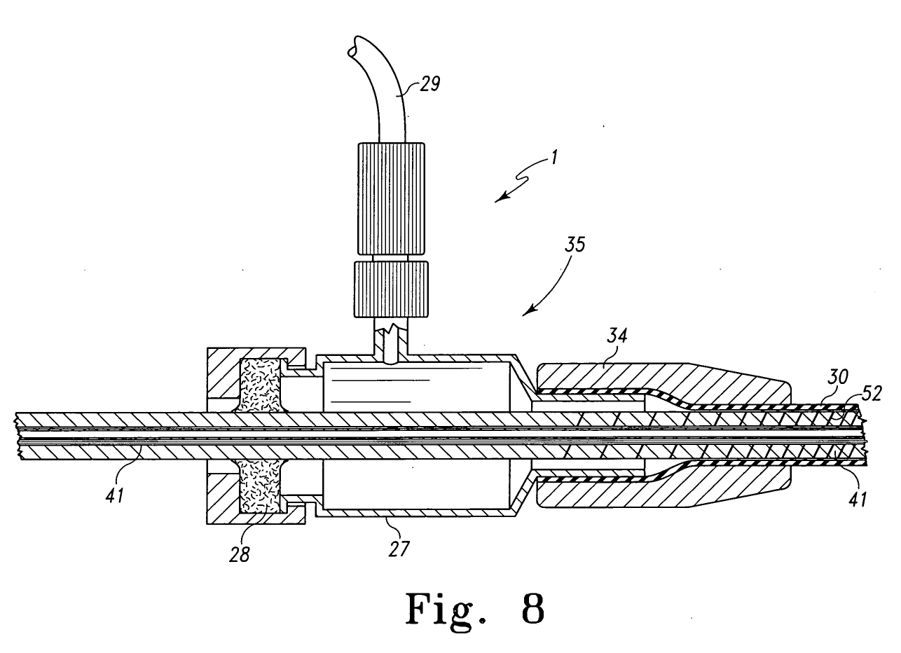

The prosthesis 20 is retained on a distal portion of the delivery and deployment device by sheath 30. Sheath 30 comprises a generally elongate tubular body having a lumen 32. The sheath 30 extends proximally to the manipulation region 1, as shown in FIG. 8. The prosthesis 20 is disposed within the lumen 32 of the sheath 30 in a radially-compressed configuration. The sheath 30 preferably comprises a flexible material so that in use it is able to negotiate complex and tortuous inner body lumina. The sheath 30 may also comprise a lubricious or slippery material to facilitate withdrawal of the sheath from the prosthesis 20 during delivery. Accordingly, the sheath 30 may comprise a biocompatible plastic such as PTFE, polyethylene, nylon, or the like.

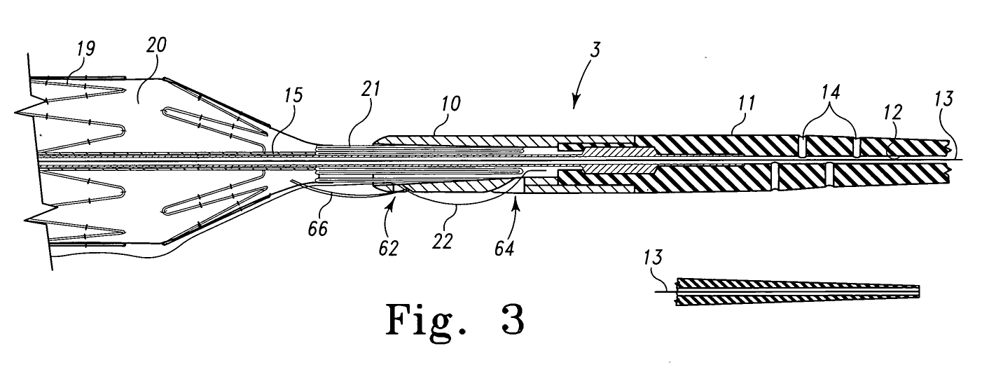

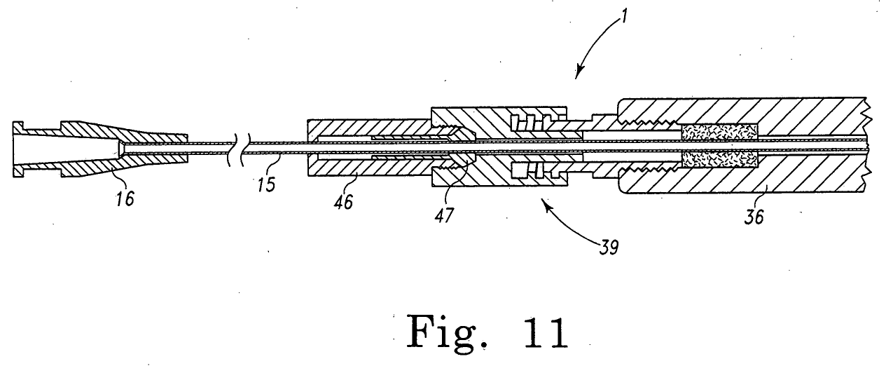

The delivery and deployment system shown in FIGS. 1-3 further comprises a thin walled tube or inner cannula 15. The inner cannula 15 is configured to receive a guide wire 13. The inner cannula 15 extends proximally to the proximal end of the delivery system. A flexible extension 11 is coupled to the distal end of the cannula 15, as shown in FIG. 3. The flexible extension 11 comprises an extension lumen 12 and a plurality of lateral apertures 14. The extension 11 is adapted for insertion into a body lumen. The cannula 15 terminates proximally at connection means 16, as shown in FIGS. 1 and 11. The connection means 16 is in fluid communication with the cannula 15, the extension lumen 12, and lateral apertures 14. The connection means 16 is adapted to accept a syringe and may be used to introduce reagents into the body lumen.

The cannula 15 is disposed within the lumen 32 of the sheath 30. The prosthesis 20 is radially retained over a distal portion of the cannula 15 by the sheath 30. The cannula 15 is preferably flexible so that the device can be advanced within a relatively tortuous vessel, such as a femoral artery or the aortic arch. The cannula 15 may comprise metal, for example aluminum, stainless steel, or nitinol. The cannula 15 is in mechanical communication with the flexible extension 11. This allows the operator to control the flexible extension 11 remotely during a procedure. For example, the operator can rotate or slide the flexible extension 11 relative to the prosthesis 20 by manipulating the cannula 15.

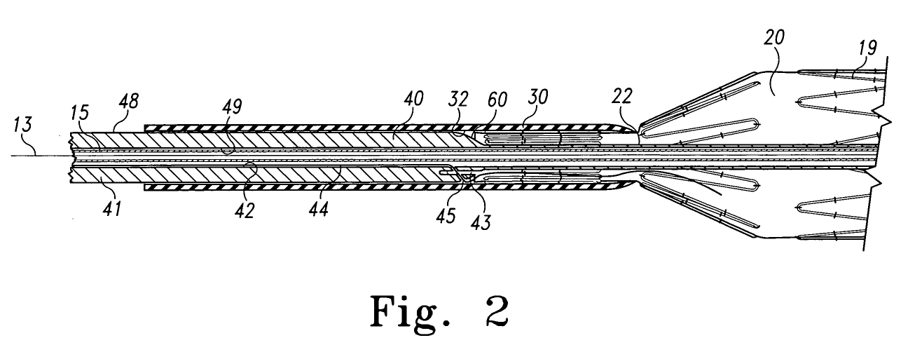

The delivery and deployment device further comprises an elongate tubular pusher 41, as shown in FIG. 2. The pusher 41 has an exterior surface 48 and a pusher lumen 42 having an interior surface 49. The cannula 15 is slidably disposed within the lumen 42 of the pusher 41. The pusher 41 extends proximally to the manipulation region 1, as shown in FIGS. 8-10. The sheath 30 is slidably disposed over a generally distal portion of the pusher 41. The delivery and deployment system further comprises haemostatic sealing means 35, shown generally in FIG. 8, for controlling blood loss through the delivery and deployment device.

As shown in FIG. 2, the distal end of the pusher 41 is disposed adjacent the proximal end of the prosthesis 20. To deploy the prosthesis 20, the operator slides the sheath 30 proximally while applying distal pressure to the pusher 41 in the user manipulation region 1. The pusher 41 comprises a blocking element 60 that prevents the prosthesis 20 from sliding proximally with the sheath 30 when the sheath is withdrawn. As a result, the sheath retracts proximally over the prosthesis, causing the prosthesis to become exposed and to expand radially outwardly.

The pusher 41 may comprise any suitable biocompatible material including metal or plastic. The pusher 41 may comprise a radiopaque material. Suitable materials include, but are not limited to aluminum, nitinol, nylon, polypropylene, and polyethylene. The pusher 41 preferably has high longitudinal column strength to ensure adequate energy transfer between the user and the prosthesis during deployment. The pusher 41 preferably has a high degree of flexibility and trackability. The pusher 41 is preferably configured so that the flexibility of the device over the proximal end of the prosthesis 20 generally matches the flexibility of the device over the distal end of the pusher 41 to avoid kinking in the transition therebetween.

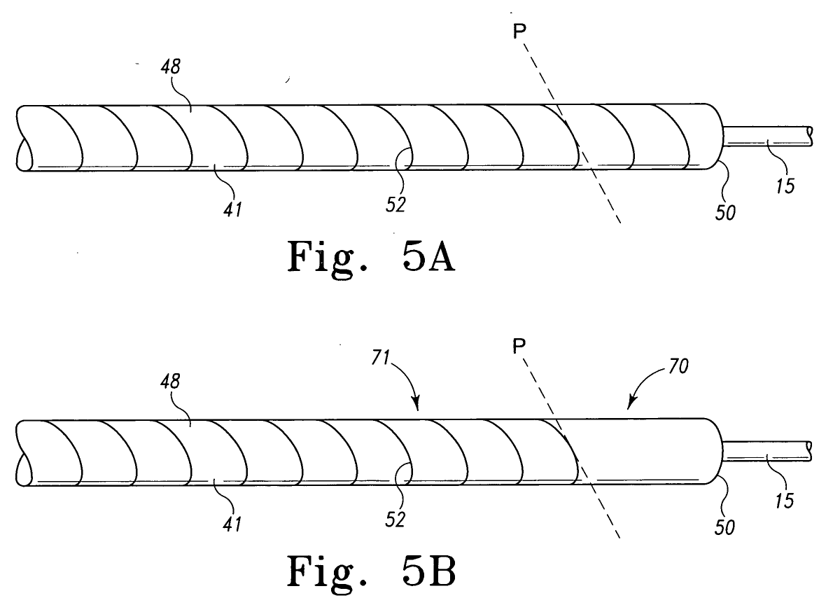

FIGS. 5A and 5B show a pusher 41 according to an aspect of the present invention. The pusher 41 is generally tubular and has a distal end 50 and a proximal end (not shown). The pusher 41 has an exterior surface 48 and an inner lumen (not shown) defining an interior surface (not shown). Inner cannula 15 is slidably disposed within the lumen 42 of the pusher 41. The pusher 41 is configured to transmit force from the user to the prosthesis 20 during use. According to an aspect of the invention, the pusher 41 comprises at least one score 52 in an exterior surface 48 thereof. The score 52 comprises a generally helical shape and extends longitudinally with the pusher 41. The score 52 comprises a thickness or depth that may be generally less than the thickness of the pusher wall, defined by the exterior and interior surfaces 48, 49. Alternatively, the score 52 may have a depth that is generally equal to the thickness of the pusher wall. The depth of the score 52 may be relatively uniform, or it may vary along the length of the pusher 41. For example, the depth of the score at its distal end may be approximately the thickness of the pusher wall, whereas the depth of the score at its proximal end may be less than the thickness of the pusher wall. In exemplary embodiments, the distal end thickness may be approximately ¾ or ½ of the thickness of the pusher wall, whereas the proximal end thickness may be approximately ½ or ¼ of the thickness of the pusher wall, respectively. It will be apparent that the flexibility of such a pusher will generally decrease proximally along the length of the score.

In general, when a tube is bent or flexed, it will tend to deform in such a way so as to minimize the overall stress on the tube. The score 52 acts as a stress riser in the pusher 41 so that when the pusher 41 is flexed, it will tend to bend at the score. The score 52 relieves tension in the tubular pusher 41 and provides a region of enhanced flexibility thereto.

The term “score” as used herein refers to a depression, cut, groove, notch, line, perforation, aperture, or the like. The term “score” may also refer to a series of depressions, cuts, grooves, notches, lines, perforations, apertures, or the like. Accordingly, a “score” is a structural element and is not limited to any particular method or process. A score may be provided by any mechanical, thermal, or chemical means known in the art. For example, a score may be provided using a knife or carbide tip, by chemical etching, by laser scoring, or by molding.

In FIG. 5A, the score 52 extends proximally from the distal end 50 of the pusher 41. In FIG. 5B, the score has a distal end that is disposed generally intermediate the proximal and distal ends of the pusher 41. The score 52 relieves tension in the pusher 41, thereby making the pusher more axially flexible.

The pusher 41 may comprise a single material along the entire length of the pusher. Alternatively, the pusher 41 may comprise a plurality of axial regions, each region comprising a different material. For example, in FIG. 5B, the pusher 41 comprises a distal tip portion 70 and a body portion 71. The distal tip portion 70 may comprise a relatively flexible material, for example a low-durometer nylon or thermoplastic elastomer and the body portion 71 may comprise a relatively rigid material, for example a high-durometer nylon or thermoplastic elastomer. The relatively flexible material in the distal tip 70 provides the pusher 41 with enhanced flexibility over a distal portion of the pusher. The relatively rigid material in the body portion 71 provides the pusher 41 with enhanced pushability. The pusher 41 may comprise a score 52 in the body portion 71. The score 52 enhances the flexibility of the body portion 71 and provides a smooth transition in flexibility between the distal tip 70 and the body portion 71.

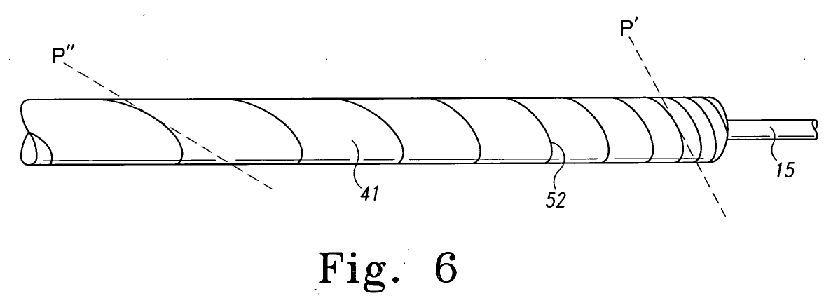

The score 52 comprises a pitch P. In general, a score with a relatively high pitch will provide a greater enhancement in flexibility to the pusher than a score with a relatively low pitch. The score 52 may comprise a generally longitudinally uniform pitch P, as shown in FIGS. 5A and 5B. Alternatively, the score 52 may have a pitch that varies proximally with the pusher 41, as shown in FIG. 6. In FIG. 6, the score 52 has a pitch P′ near the distal end of the pusher 41 that is greater than a pitch P″ at a position proximal of the distal end. As shown, the pusher 41 has a score 52 that has a proximally-decreasing pitch, and therefore a distally-increasing flexibility. The pusher 41 may alternatively have a score 52 with a proximally-increasing pitch P, and therefore a distally-decreasing flexibility.

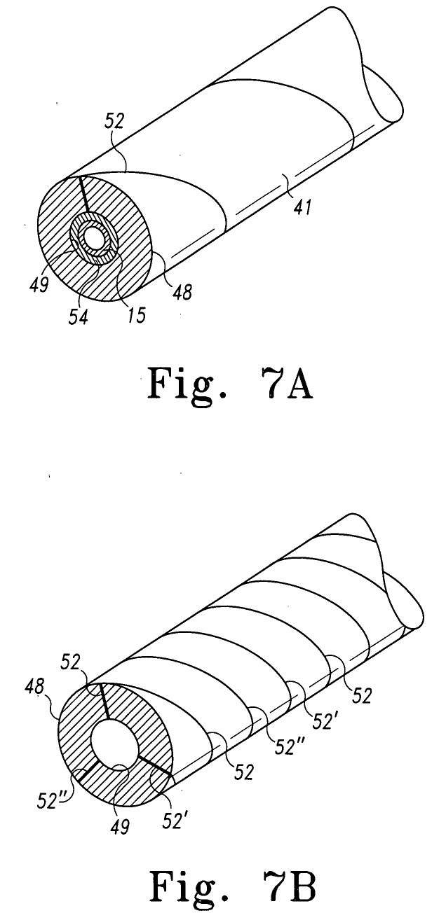

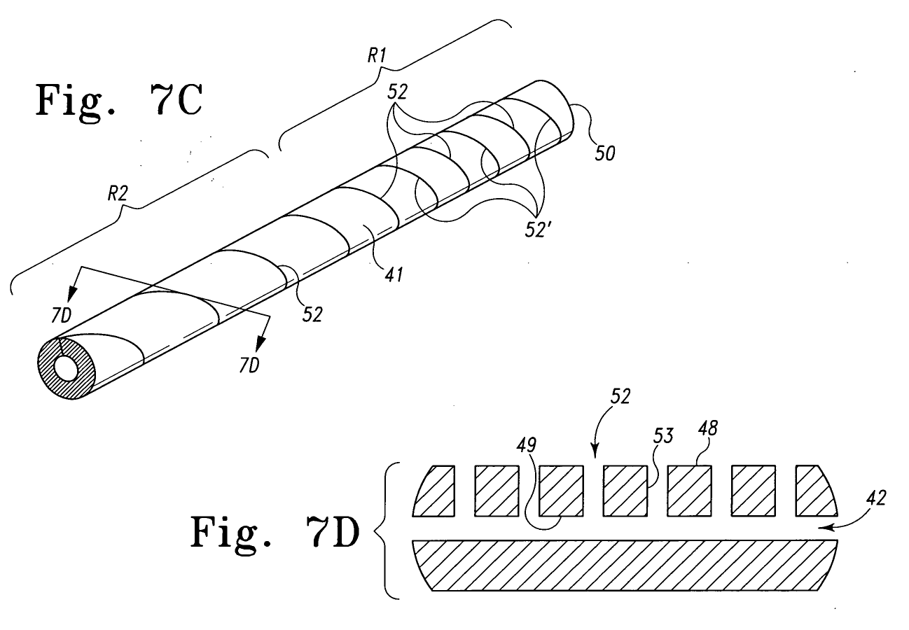

FIGS. 7A-7D illustrate additional aspects of the present invention. In FIG. 7A, the pusher 41 comprises a single helical score 52 that extends longitudinally with the pusher 41. As shown in FIG. 7A, the delivery and deployment device may comprise an inner sheath 54. The inner sheath 54 is radially disposed within the lumen 49 of the pusher 41 and extends between the distal and the proximal end. The inner sheath 54 is fixedly attached to the pusher 41. The inner sheath 54 may be attached to the pusher 41 at a single point or at a plurality of points along the length of the pusher. Alternatively, the inner sheath 54 and the pusher 41 may be uniformly bonded along the length of the pusher 41. The inner sheath 54 may be formed independently of the pusher 41 and may be attached using, for example, a biocompatible adhesive or a thermal bonding technique generally known in the art. Alternatively, the inner sheath 54 may be formed generally concurrently with the pusher, for example by co-injection molding or by co-extrusion. The inner sheath 54 preferably comprises a lubricious material to decrease the friction between the cannula 15 and the pusher 41. The inner sheath 54 preferably comprises a flexible material that is more flexible than the pusher 41 so that it will not significantly impact the flexibility of the system. Accordingly, the inner sheath 54 may comprise a flexible plastic such as PTFE, polyethylene, or a synthetic rubber or a thermoplastic elastomer.

The inner sheath 54 provides a barrier between the inner cannula 15 and the pusher 41. As stated previously, the score 52 serves as a concentration site for bending stresses imposed on the pusher 41. When the pusher 41 is flexed, the bending stress is transmitted through the pusher 41 to the cannula 15. When the pusher 41 comprises a score 52, the stress is distributed over the pusher 41 in discrete highly concentrated regions generally associated with the score 52. These regions of highly concentrated stress can promote damage to the cannula, including kinking. The inner sheath 54 may provide an absorptive layer between the pusher 41 and the cannula 15 and dispers the bending stresses longitudinally along the delivery and deployment device.

The inner sheath 54 may also provide structural support and integrity to the pusher 41. When the sheath 30 is slid proximally in relation to the pusher 41, the coils created by the helical score 52 are held together in compression. However, when the sheath 30 is slid distally in relation to the pusher 41, for example during recapture of the prosthesis 20 or during withdrawal of the delivery and deployment device from the body lumen, the pusher 41 is exposed to tensile loading which will promote longitudinal expansion of the pusher 41 and expansion of the coils. The inner sheath 54 supports and reinforces the pusher 41 and prevents the pusher 41 from longitudinally expanding.

In FIG. 7B, the pusher 41 comprises a plurality of helical scores 52, 52′, 52″. Scores 52, 52′, 52″ each extend longitudinally with the pusher 41. As shown in FIG. 7B, the scores 52, 52′, 52″ may be positioned radially symmetrically about the pusher 41. For example, the pusher 41 may comprise three helical scores, each disposed 120 degrees from the other. Alternatively, the pusher 41 could comprise four helical scores, each disposed 90 degrees from the other. It should be readily apparent that the pusher 41 could comprise any number of scores according to the particular application, and that the scores 52 could be positioned radially symmetrically or radially non-symmetrically about the pusher 41. In general, the flexibility of the pusher 41 will be directly proportional to the number of scores 52 in the pusher 41 as well as the positioning of the scores 52 about and along the pusher 41.

FIG. 7C shows another pusher 41 according to an aspect of the present invention. The pusher 41 comprises two scores 52, 52′. The scores 52, 52′ are disposed 180 degrees from each other. Score 52 is longer than score 52′. The pusher 41 comprises a first region R1 generally associated with score 52′ and a distal portion of score 52. The pusher 41 further comprises a second region R2 generally associated with a proximal portion of score 52. Because the first region R1 has a higher score density than the second region R1, the first region R1 is more axially flexible than the second region R2.

As shown in FIG. 7C, the scores 52, 52′ may be longitudinally coterminous. That is, the scores may have ends that terminate at generally the same longitudinal position on the pusher 41. In FIG. 7C, scores 52, 52′ have distal ends that terminate at the distal end 50 of the pusher 41. Alternatively, the scores could have coterminous proximal ends, or the distal end of one of the scores may be coterminous with the proximal end of another score.

FIG. 7D illustrates a further aspect of the present invention. As shown in FIG. 7D, the score 52 may comprise a series of discrete score elements. Such a score 52 may be provided by a laser scoring process. The score 52 is defined by a plurality of perforations 53 in the exterior surface 48 of the pusher 41. The perforations are provided by discrete energy bursts emitted by the laser and absorbed by the pusher 41. The perforations 53 are arranged in a generally helical pattern along the pusher 41. The score 52 provides a stress-concentrator in the pusher 41, thereby enhancing pusher flexibility.

FIG. 8 shows a portion of the delivery and deployment device around the proximal end of the sheath 30. The device may comprise a haemostatic device 35 that provides a haemostatic seal between the sheath 30 and the pusher 41. The haemostatic device 35 may comprise a haemostatic seal 27. In FIG. 8, a clamping collar 34 clamps the sheath 30 to the haemostatic device 35. The haemostatic seal 27 may include a silicone seal ring 28. The silicone seal ring 28 forms a haemostatic seal around the pusher 41. The haemostatic device 35 may also include a side tube 29. The side tube 29 facilitates the introduction of medical reagents between the pusher 41 and the sheath 30.

The haemostatic device 35 controls blood loss through the delivery and deployment device distal of the haemostatic seal 27. Blood loss can be controlled proximal of the haemostatic seal 27 in several ways. First, the pusher 41 may provide a haemostatic seal. Accordingly, the portion of the pusher 41 proximal of the haemostatic seal 27 may be provided without a score 52. Alternatively, the portion of the pusher 41 proximal of the haemostatic seal 27 may comprise a score 52 that has a thickness or depth that is generally less than the wall thickness of the pusher to prevent blood leakage through the pusher 41. Alternatively, the system may comprise an inner sheath 54, as described above, wherein the inner sheath is fluid-impermeable. The fluid-impermeable inner sheath 54 provides a haemostatic seal over the entire pusher, including the portion of the pusher 41 proximal of the haemostatic seal 27. Accordingly, the portion of the pusher 41 proximal of the haemostatic seal 27 may comprise a score 52 having a thickness that is generally equal to the wall thickness of the pusher.

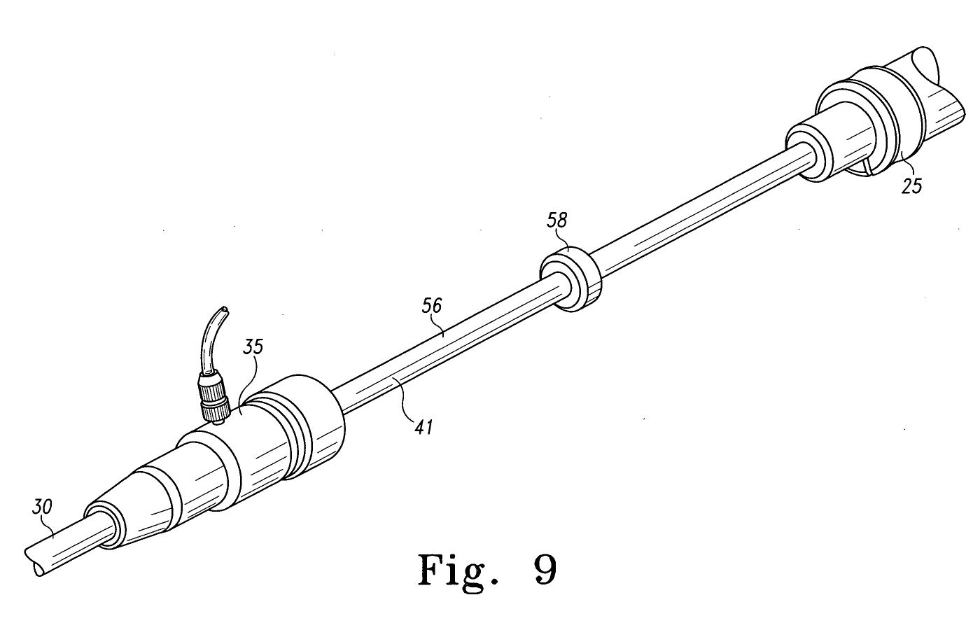

The delivery and deployment device may optionally comprise a limiting member 58, disposed on the proximal portion of the pusher 41, that limits the proximal travel of the sheath 30 during deployment. As shown in FIG. 9, the limiting member 58 may comprise a radial projection in the exterior surface 48 of the pusher 41 that is configured to limit the proximal movement of the sheath 30. As the sheath 30 slides proximally over the prosthesis 20 and the pusher 41, the sealing means 35 slides towards limiting member 58 over the slide region 56. The sheath 30 is prevented from sliding proximally when the sealing means 35 engages limiting member 58. The position of limiting member 58 may be configured so that the length of the slide region 56 is generally equal to or less than the distance between the distal end of the sheath 30 and the distal pusher end 50. In this way, the distal end 50 of the pusher 41 will always be contained within the sheath 30.

As shown in FIG. 2, the delivery and deployment device may optionally comprise proximal and/or distal retention and release mechanisms for radially and/or axially retaining proximal and distal ends of the prosthesis 20. FIG. 2 illustrates a proximal prosthesis retention mechanism. The proximal retention section 40 radially and axially retains a proximal end of the prosthesis 20 during the procedure. The proximal retention section 40 may comprise the pusher, as shown in FIG. 2. Alternatively, the proximal retention section 40 may comprise a separate body coupled to the pusher 41.

The proximal end of the prosthesis 20 comprises an aperture defining a loop 43. A proximal trigger wire 44 extends through the loop 43 and through an aperture 45 in the proximal attachment section 40 into an annular region between the thin walled tube 15 and the thick walled tube 41. The proximal trigger wire 44 extends proximally through the delivery and deployment device from the proximal retention section 40 to the release wire actuation section located in the external manipulation section 1 (see FIG. 1). The trigger wire 44 couples the proximal end of the prosthesis 20 to the proximal retention section 40 during deployment to limit axial displacement of the prosthesis. The prosthesis 20 can be selectively released into the body lumen by disengaging the trigger wire 44 from the loop 43.

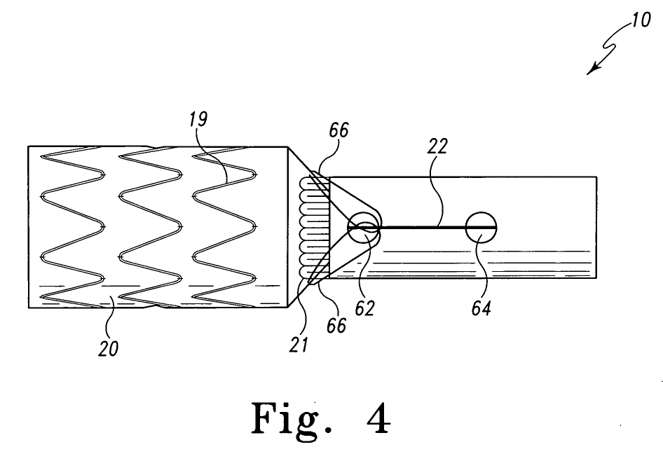

FIGS. 3 and 4 illustrate a distal retention mechanism. The distal attachment region 3 includes a retention device 10. The retention device 10 holds the distal end of the bare wire stent 21 in a radially compressed state. The retention device 10 may further comprise means for axially retaining the stent 21. Accordingly, the stent 21 may be retained in the retention device 10 by suture loops 66 and a distal trigger wire 22. The suture loops 66 and distal trigger wire 22 removably couple the stent 21 to the retention device 10.

FIG. 4 is a plan view of the retention device 10 showing the prosthesis 20 partially deployed, with the bare wire stent 21 still retained in a compressed state. The distal retention device 10 includes apertures 62 and 64 to accommodate the distal trigger wire 22. The suture loops 66 are coupled to the body of the prosthesis 20, and hold the stent 21 in the retention device 10 until the trigger wire 22 is removed. While the trigger wire 22 is in place, the suture loops 66 prevent the retention device 10 and the prosthesis 20 from separating. The trigger wire 22 retains the suture loops 66 against an outer surface of the retention device 10. The distal trigger wire 22 extends proximally through the delivery and deployment device from the distal retention device 10 to a release wire actuation section located in the manipulation section 1 (see FIG. 1).

As shown in FIG. 4, the suture loops 66 are attached to opposing sides of the prosthesis 20, for example separated by 90 to 180 degrees. The suture loops 66 are generally inelastic and do not stretch. Since the suture loops 66 do not stretch, they provide opposing torques, thereby preventing the prosthesis 20 from rotating within the retention device 10. This configuration differs from delivery and deployment devices that have a single point of attachment. Such devices may allow the stent to rotate within the retention device and lead to entanglement of the stent's struts. When the trigger wire 22 is removed, the suture loops 66 are free to move. The retention device 10 may then be released from the bare wire stent 21 by sliding the retention device 10 distally away from the prosthesis 20.

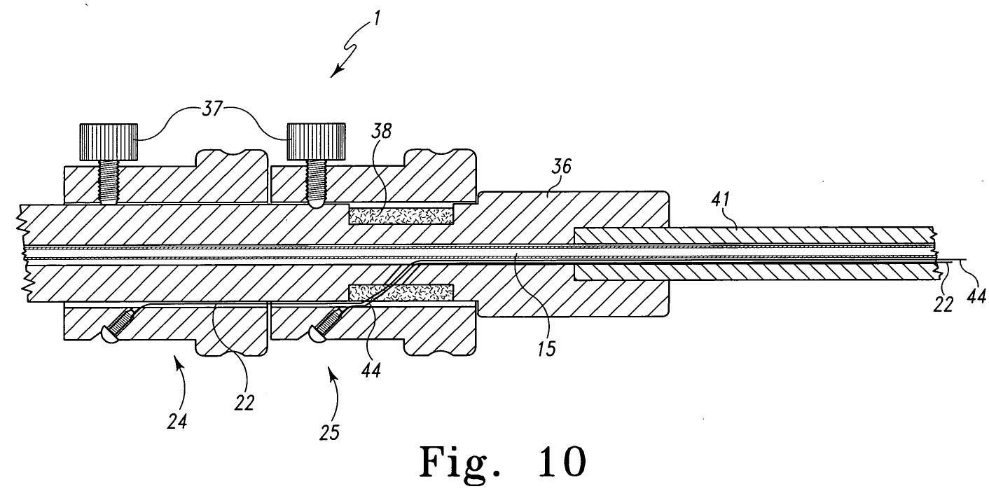

As shown in FIG. 10, the distal trigger wire 22 extends through the annular space between the pusher 41 and the cannula 15 to the manipulation region 1. The distal trigger wire 22 exits the annular space at a distal wire release mechanism 24. The bare wire stent 21 is released by retracting the sheath 30, removing the trigger wire 22, and then sliding the distal attachment region 3, including the retention device 10, distally away from the stent 21. Once the retention device 10 has cleared the bare wire stent 21, the stent 21 will expand. The suture loops 66, the trigger wire 22, and the distal wire release mechanism 24 form a control member to selectively release the retention device 10 from the prosthesis 20 by holding the self-expanding stent 21 in the retention device 10 until the prosthesis 20 is positioned at a desired site in the lumen.

The release wire actuation section has a body 36 that is mounted onto a proximal portion of the pusher 41, as shown in FIG. 10. The cannula 15 passes through the body 36. The proximal wire release mechanism 25 is mounted for slidable movement on the body 36. A clamping screw 37 prevents inadvertent early release of the proximal end of the prosthesis 20. Similarly, the distal wire release mechanism 24 is mounted for slidable movement on the body 36. A clamping screw 37 prevents inadvertent early release of the bare wire stent 21.

The proximal trigger wire 44 extends through the annular space between the pusher 41 and the cannula 15 to the manipulation region. The proximal trigger wire 44 exits the annular space at a proximal wire release mechanism 25. The proximal trigger wire 44 and the proximal wire release mechanism 25 form a control member to selectively release the proximal retention section 40 from the prosthesis when the prosthesis is positioned at a desired site in the lumen.

The positioning of the distal and proximal wire release mechanisms 24 and 25 is such that the distal wire release mechanism 24 must be moved before the proximal wire release mechanism 25 can be moved. Therefore, the proximal end of the prosthesis 20 cannot be released until the bare wire stent 21 has been released and anchored to the lumen. A haemostatic seal 38 is provided so the release wire 44 can extend out through the body 36 to the release mechanism 25 without unnecessary blood loss during the medical procedure.

FIG. 11 shows a proximal portion of the external manipulation section 1. A pin vise 39 is mounted onto the proximal end of the body 36. The pin vise has a screw cap 46. When screwed in, the vise jaws 47 clamp against (engage) the cannula 15. When the vise jaws 47 are engaged, the cannula 15 can only move with the body 36, and hence the cannula 15 can only move with the pusher 41 (not shown). With the screw cap 46 tightened, the entire assembly, except for the sheath 30, can be moved as one.

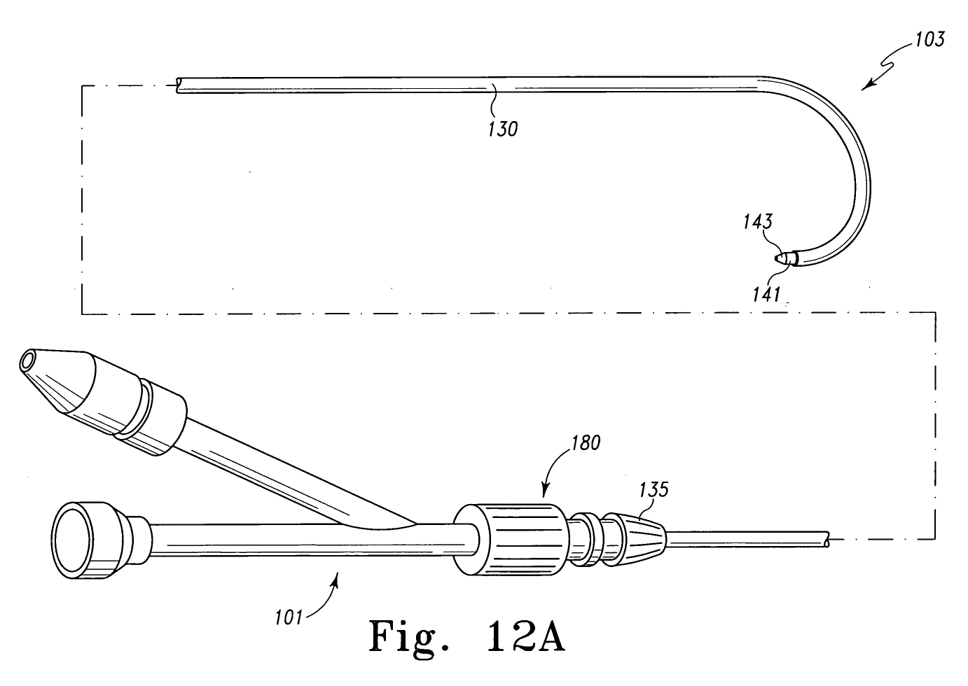

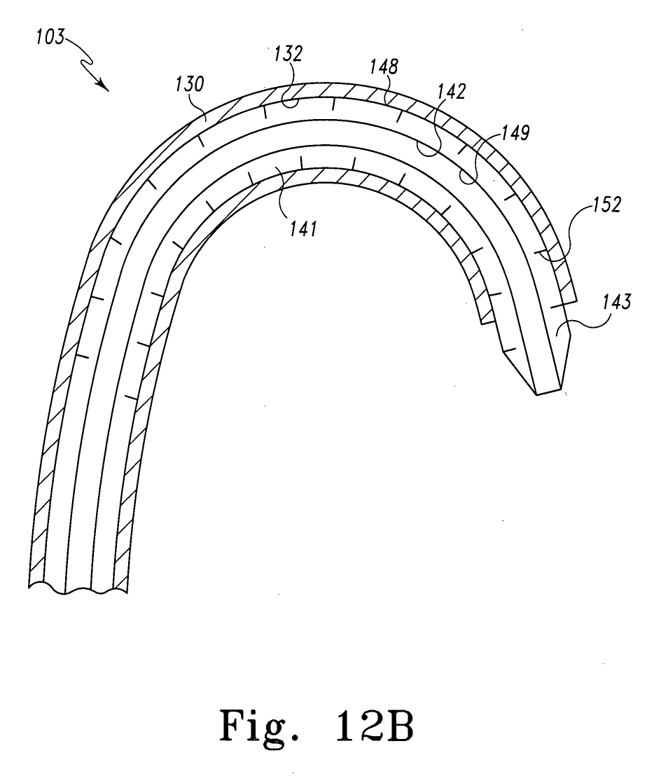

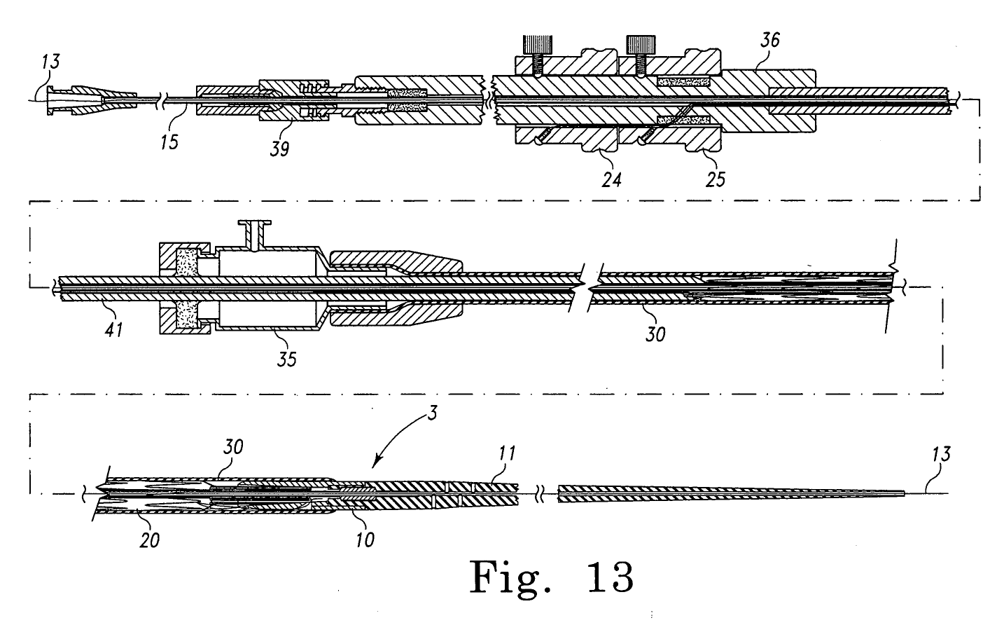

FIGS. 12A and 12B show another delivery system for an intraluminal medical device such as a prosthesis, a balloon catheter, a diagnostic catheter, or the like. The system includes a proximal external manipulation section 101 and a distal positioning mechanism 103. During a procedure, the distal positioning mechanism 103 will travel through the lumen to a desired deployment site, whereas the external manipulation section 101 is acted upon by a user outside of the patient to manipulate the delivery system.

The delivery system comprises a sheath 130 and a pusher 141 that is slidably and removably disposed within a lumen 132 of the sheath. The pusher 141 has an exterior surface 148 and may include a lumen 142 defining an interior surface 149, for example, for receiving a guide wire (not shown). The sheath 130 and the pusher 141 extend proximally to the external manipulation section 101. The pusher 141 provides radial and longitudinal support to the sheath 130 so that the distal end of the sheath can be intraluminally delivered to a desired location in a body lumen. Once the sheath 130 is in the desired location, the pusher 141 may be removed and additional interventional catheter devices may be delivered and deployed through the sheath lumen 132.

In the embodiment shown in FIGS. 12A and 12B, the distal end of the pusher 141 comprises a dilator 143. During delivery of the system, the dilator 143 preferably extends distally from the sheath lumen 132 and facilitates dilation of constricted vessels as the system travels within a body lumen. The dilator 150 is preferably tapered and provides a generally smooth transition between the pusher 141 and the distal end of the sheath 130.

As shown in FIG. 12A, a haemostatic device 135, such as the device shown in FIG. 8, may be attached to the sheath 130 for providing a haemostatic seal between the sheath 130 and the pusher 141. A coupling mechanism 180, for example, a screw cap may be provided for selectively coupling the sheath 130 and the pusher 141 to prevent relative movement therebetween. The coupling mechanism 180 may be detached, for example, during insertion or retraction of the pusher 141 from the sheath lumen 132. The coupling mechanism 180 may be attached, for example, during intraluminal delivery of the system.

The delivery system may have a generally straight contour along its entire length or it may comprise an arcuate or curved contour. In the embodiment shown in FIGS. 12A and 12B, a distal portion of the delivery system has a pre-set arcuate contour, whereas a proximal portion has a generally straight contour. The proximal portion is preferably relatively rigid and has a high degree of pushability to facilitate delivery of the sheath 130 into the body lumen. Alternatively, the distal portion is preferably relatively flexible and has a high degree of trackability to facilitate tracking of the system through tortuous body luminae.

In prior art systems where the sheath has an arcuate contour, the sheath tends to straighten during insertion and retraction of the pusher, which can potentially cause bending and kinking of the sheath. This is true, even where the pusher has a corresponding pre-set contour. According to an aspect of the invention, the pusher 141 may be provided with a relatively high flexibility region over a portion corresponding with the arcuate contour to facilitate tracking during insertion and retraction of the pusher 141 within the sheath lumen 132.

The pusher 141 may be configured as described throughout the specification. As shown in FIG. 12B, the pusher 141 may include at least one generally helical score 152 in an exterior surface 148 thereof to provide flexibility to the pusher. The score or scores may be configured as described throughout the specification. For example, the at least one score 152 may comprise a uniform or varying thickness or depth and may comprise a uniform or varying pitch. In the embodiment shown in FIG. 12B, the score 152 has a depth that is less than the thickness of the pusher wall defined by the interior and exterior surfaces of the pusher 141.

The at least one score 152 preferably extends along the length of the arcuate contour, and more preferably extends proximally from the distal end of the pusher 141 over the length of the arcuate contour, as shown in FIG. 12B. It will be appreciated, however, that one or more scores 152 may alternatively, or additionally be disposed at any location along the pusher 141, as required.

The various stages of deployment of a prosthesis 20 using a delivery and deployment device of the present invention will now be explained with reference to FIGS. 13 through 17. A guide wire 13 is introduced, for example, into the femoral artery and is advanced until the tip of the guide wire 13 is beyond the region into which the prosthesis 20 is to be deployed. The delivery assembly is then inserted through the femoral artery over the guide wire 13, and positioned by radiographic techniques, generally known in the art. At this stage, the ends of the prosthesis 20 are retained by the distal and proximal retaining assemblies respectively and the sheath 30 is disposed over and covers the length of the prosthesis 20.

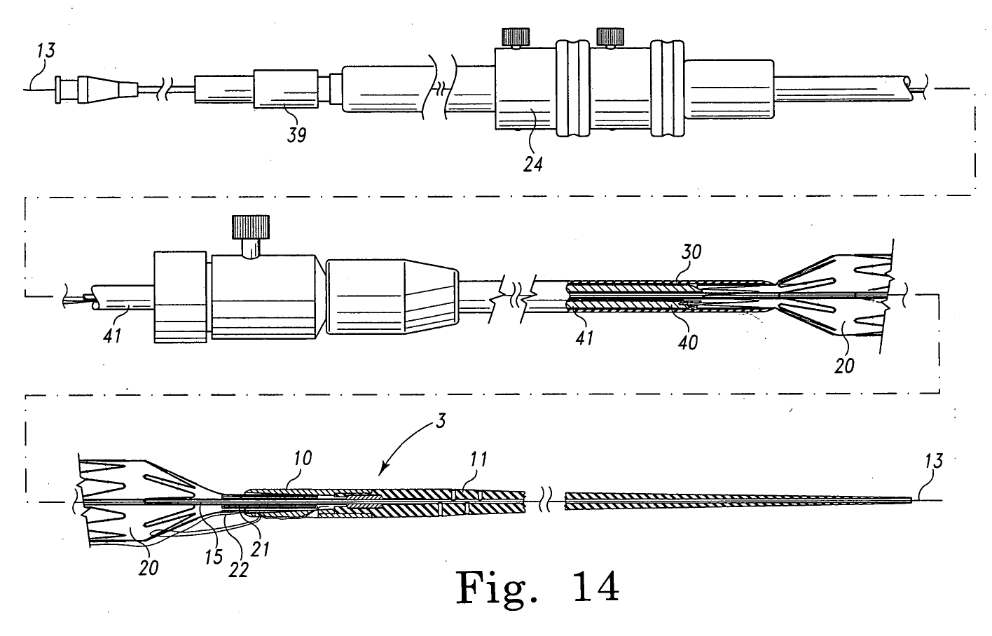

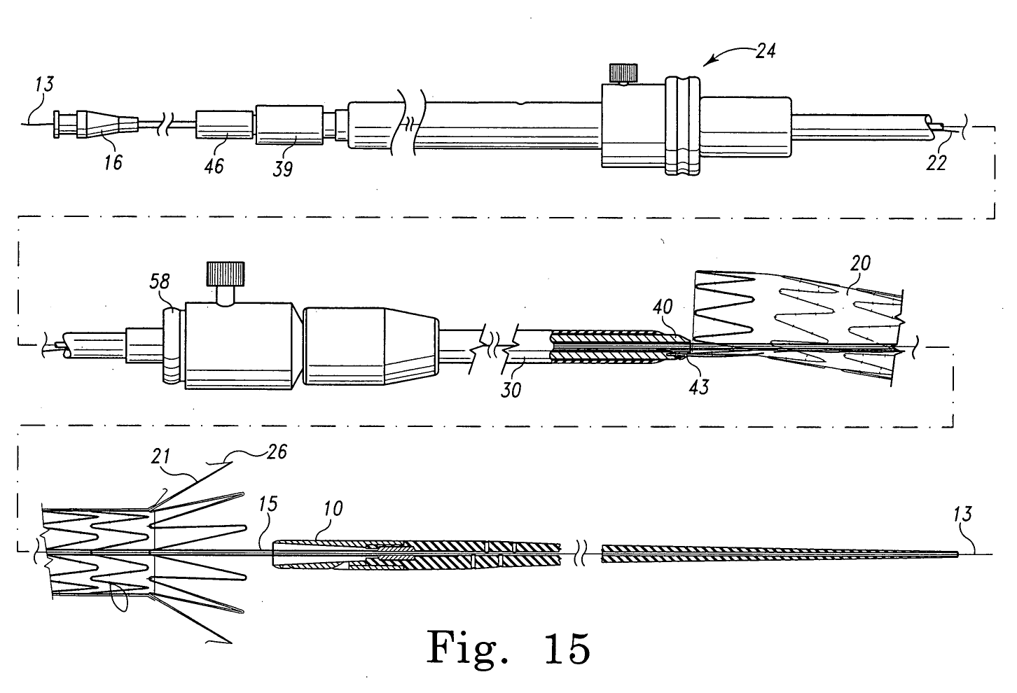

In FIG. 13, the delivery and deployment assembly is shown fully assembled and ready for introduction into a patient. The ends of the prosthesis 20 are retained by the distal and proximal retaining assemblies respectively, while the sheath 30 compresses the middle portion of the prosthesis intermediate the ends. Once the delivery and deployment device is in a desired position for deployment of the prosthesis 20, the sheath 30 can be withdrawn to just distal of the proximal attachment section 40, as shown in FIG. 14. This action exposes the middle portion of the prosthesis 20 so that the middle portion can expand radially outwardly. The bare wire stent 21, however, is still axially and radially retained by the retention device 10. The proximal end of the prosthesis 20 is still radially and axially retained by the proximal retention section 40.

Next, the pin vise 39 is released to allow small movements of the cannula 15 with respect to the pusher 41. In this way, the prosthesis 20 may be lengthened or shortened or rotated or compressed for accurate placement in the desired location within the lumen. X-ray opaque markers (not shown) may be placed along the prosthesis 20 to assist with placement of the prosthesis.

In FIG. 15, the distal trigger wire 22 has been removed, allowing the retention device 10 to be separated from the bare wire stent 21, as explained above. At this stage, the distal trigger wire release mechanism 24 and the distal trigger wire 22 can be removed completely. The screw cap 46 of the pin vise 39 has been loosened so that the cannula 15 can be pushed in a distal direction to move the retention device 10 in a distal direction with respect to the stent 21. When the retention device 10 no longer surrounds the self-expanding stent 21 at the distal end of the prosthesis 20, the self-expanding stent 21 expands. When the self-expanding stent 21 expands, the barbs 26 grip into the walls of the lumen to hold the proximal end of the prosthesis 20 in place.

At this point, the proximal end of the prosthesis 20 is still retained by the proximal retention section 40 with the loop 43 retained therein. The sheath 30 is withdrawn to proximal of the proximal retention section 40 to allow the proximal end of the prosthesis 20 to expand. The limiting member 58 limits the travel of the sheath 30. At this point, the proximal end of the prosthesis may still be moved. Consequently, the prosthesis 20 can still be rotated or lengthened or shortened or otherwise moved for accurate positioning. Where the prosthesis 20 to be deployed is a bifurcated graft, the movement at this stage may ensure that the shorter leg is directed in the direction of the contra-iliac artery.

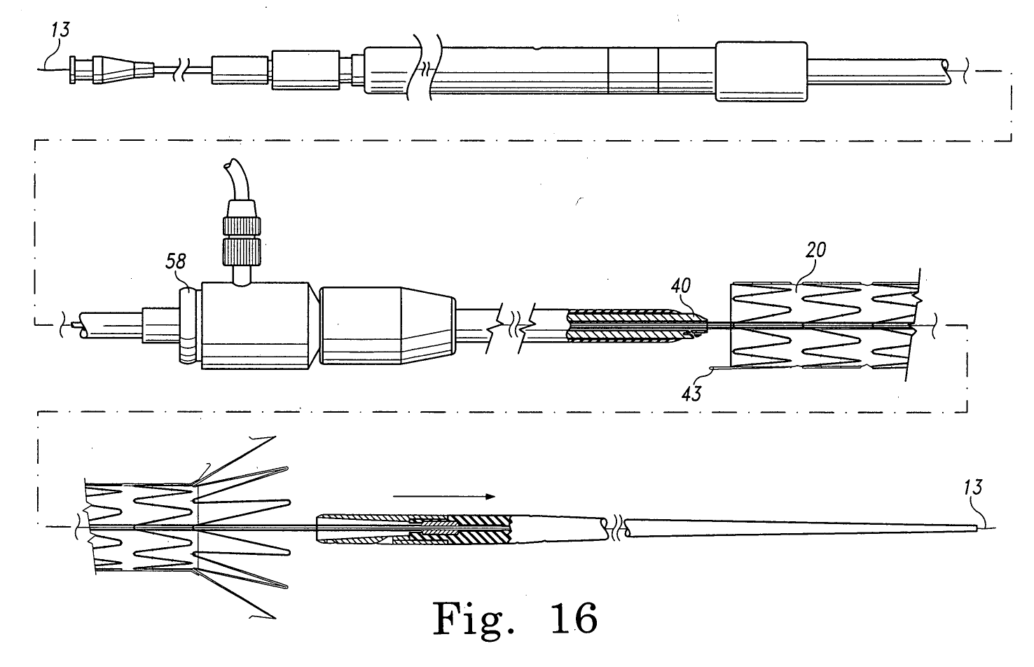

In FIG. 16, the proximal end of the prosthesis 20 has been released by the removal of the proximal trigger wire 44. At this stage, the proximal trigger wire release mechanism 25 and the proximal trigger wire 44 can be removed completely. This removal may be accomplished by passing the proximal wire release mechanism 25 over the pin vise 39 and the connection means 16, thereby disengaging the trigger wire 44 from the prosthesis 20. The prosthesis is now free to expand to the walls of the vessel.

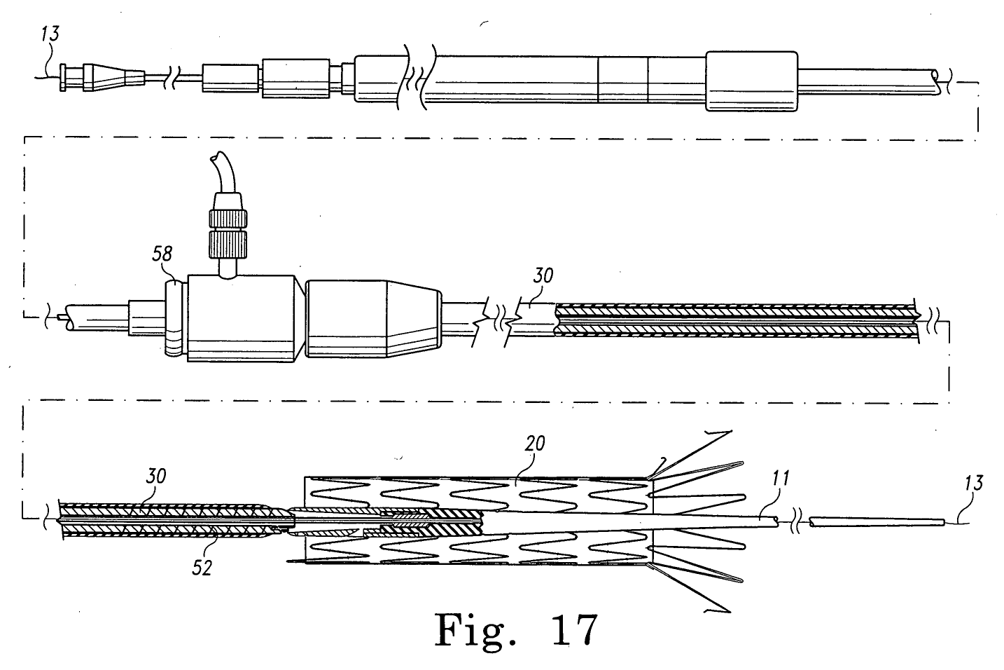

The device is now ready to be removed. The screw cap 46 of the pin vise 39 is loosened so that the cannula 15 can be moved. The cannula 15 is pulled in a proximal direction to move the retention device 10 until it comes into contact with the proximal retention section 40, as shown in FIG. 17. The pin vise 39 is then tightened so that the retention device 10 is in fixed relation to the proximal retention section 40. The entire system, including the sheath 30 may now be removed from the body lumen by pulling proximally on the system. Alternatively, the inner cannula 15, the pusher 41, and the flexible extension 11 may be removed from the sheath 30. This is done by pulling the cannula 15 proximally in relation to the sheath 30. The distal retention device 10 provides a compressive force against the pusher 41, preventing the pusher 41 from elongating or unraveling during withdrawal.

While various embodiments of the invention have been described, it will be apparent to those of ordinary skill in the art that many more embodiments and implementations are possible within the scope of the invention. Furthermore, although various indications have been given as to the scope of this invention, the invention is not limited to any one of these but may reside in two or more of these combined together. Accordingly, the invention is not to be restricted except in light of the attached claims and their equivalents.

1. A delivery system for an intraluminal medical device, the system comprising:
an elongate tubular sheath having a proximal end, a distal end, and a sheath lumen;
an elongate tubular pusher slidably disposed within the sheath lumen, the pusher having a proximal end, a distal end, an exterior surface, and a lumen defining an interior surface;
wherein the pusher further comprises at least one generally helical score in the exterior surface, the score providing enhanced flexibility to the pusher.

2. The system according to claim 1, further comprising a radially-expandable intraluminal prosthesis disposed in a compressed configuration within a distal portion of the sheath lumen, wherein the pusher is configured to push the prosthesis distally within the sheath lumen when the sheath is slid proximally in relation thereto.

3. The system according to claim 1, wherein the distal end of the pusher comprises a dilator having a tapered distal end.

4. The system according to claim 1, wherein the score has a first pitch at a first longitudinal position along the pusher and a second pitch at a second longitudinal position along the pusher, the second pitch being greater than the first pitch.

5. The system according to claim 1, wherein the at least one score has a longitudinally-uniform pitch.

6. The system according to claim 1, wherein the at least one score has a longitudinally-varying pitch.

7. The system according to claim 6, wherein the pitch decreases proximally with the pusher.

8. The system according to claim 1, wherein the at least one score has a depth that is less than or equal to the thickness of the pusher wall defined by the interior and exterior surfaces of the pusher.

9. The system according to claim 1, wherein the at least one score extends proximally from the distal end of the pusher.

10. The system according to claim 1, wherein the pusher comprises a plurality of helical scores positioned symmetrically about the pusher.

11. The system according to claim 1, wherein the pusher comprises a first helical score having a proximal end and a distal end and a second helical score having a proximal end and a distal end.

12. The system according to claim 11, wherein the first score has a length that is greater than a length of the second score.

13. The system according to claim 11, wherein at least one of the distal end and the proximal end of the first score is coterminous with at least one of the distal end and the proximal end of the second score.

14. The system according to claim 1 further comprising an elongate inner sheath fixedly disposed within the pusher lumen between the proximal and distal pusher ends.

15. The system according to claim 14, wherein the inner sheath comprises a flexible plastic material and the pusher comprises a material that is more rigid than the inner sheath material.

16. The system according to claim 1, wherein the pusher comprises a first material along a first portion thereof, and a second material along a second portion thereof, the first material being more flexible than the second material, and wherein the second portion of the pusher comprises at least one generally helical score in the exterior surface, the score providing enhanced flexibility to the second portion of the pusher.

17. The system according to claim 1 further comprising a limiting member for limiting the proximal displacement of the sheath relative to the pusher, thereby limiting distal retraction of the pusher from the sheath lumen.

18. The system according to claim 1 further comprising a haemostatic seal for controlling blood loss through the pusher.

19. The system according to claim 1 further comprising a guide cannula having a proximal end and a distal end, the guide cannula disposed within the lumen of the pusher and configured to receive the expandable prosthesis over a distal portion thereof.

20. The system according to claim 1:
wherein the pusher comprises a plurality of helical scores positioned symmetrically about the pusher, the plurality of scores including a first helical score having a proximal end and a distal end, and a second helical score having a proximal end and a distal end;
wherein at least one of the first and second scores extends proximally from the distal end of the pusher, has a depth that is less than or equal to the thickness of the pusher wall defined by the interior and exterior surfaces of the pusher, and has one of a longitudinally-uniform pitch and a longitudinally-varying pitch;
wherein the first score has a length that is greater than a length of the second score and wherein at least one of the distal end and the proximal end of the first score is coterminous with at least one of the distal end and the proximal end of the second score;
wherein the pusher comprises a first material along a first portion thereof, and a second material along a second portion thereof, the first material being more flexible than the second material, and wherein the second portion of the pusher comprises at least one generally helical score in the exterior surface, the score providing enhanced flexibility to the second portion of the pusher; and
wherein the system further comprises:
a radially-expandable intraluminal prosthesis disposed in a compressed configuration within a distal portion of the sheath lumen, wherein the pusher is configured to push the prosthesis distally within the sheath lumen when the sheath is slid proximally in relation thereto;
an elongate inner sheath fixedly disposed within the pusher lumen between the proximal and distal pusher ends, the inner sheath comprising a flexible plastic material, and wherein the pusher comprises a material that is more rigid than the inner sheath material;
a limiting member for limiting the proximal displacement of the sheath relative to the pusher, thereby limiting distal retraction of the pusher from the sheath lumen;
a haemostatic seal for controlling blood loss through the pusher; and
a guide cannula having a proximal end and a distal end, the guide cannula disposed within the lumen of the pusher and configured to receive the prosthesis over a distal portion thereof.
